(19) United States
(12) Patent Application Publication
(10) Pub. No.: US 2026/0102050 A1
(43) Pub. Date: Apr. 16, 2026

(54) DRYING AND SANITIZING SYSTEM

(71) Applicant: Patricia OSWALD, Libertyville, IL (US)

(72) Inventor: Patricia OSWALD, Libertyville, IL (US)

(21) Appl. No.: 19/115,731

(22) PCT Filed: Sep. 26, 2023
(86) PCT No.: PCT/US2023/075109

§ 371 Date: Mar. 26, 2025

Related U.S. Application Data
(60) Provisional application No. 63/409,978, filed on Sep. 26, 2022.

(51) Int. Cl. A47L 19/00; A61L 2/104; A61L 103/00; F26B 9/00

(52) U.S. Cl. CPC A47L 19/00; A61L 2/104; F26B 9/003; A61L 2103/23; A61L 2202/11; A61L 2202/16

(57) ABSTRACT

A drying system includes a base, a fan assembly within the base capable of creating an air flow, and at least one elongate, hollow drying arm configured to receive an article to be dried, in communication with the air flow, wherein the at least one drying arm includes at least one aperture allowing said air flow to escape therefrom.

### REFERENCE TO RELATED APPLICATIONS

This application claims priority to and the benefit under 35 USC § 119(e) of U.S. Provisional No. 63/409,978 , filed on Sep. 26, 2022, the contents of which are incorporated by reference in their entirety as if fully set forth herein.

### TECHNICAL FIELD

This disclosure relates to systems and methods for drying reusable household items. In particular, this disclosure relates to systems and methods for a drying rack that combines a forced air feature and optional sterilizing lights so that bottles, bags and other reusable items can be dried, sanitized, and re-used.

### BACKGROUND

Plastic products such as bottles, bags and other conveniences are widely believed to be one of the greatest contributors to global pollution. Plastic bottles are so ubiquitous that it is believed that 20,000 such items are bought every second around the world, with annual consumption reaching half a trillion bottles in 2021. Each plastic bottle takes between 400-1000 years to decompose in a landfill; the United States alone uses an average over 58 million resealable plastic bags every year and over 5 trillion pieces of plastic waste are thought to exist in the oceans.

The convenience of disposable plastic items is countered by the environmental devastation they cause, since most of these items can last for decades, if not centuries in landfills and, more problematically, oceans and other bodies of water. Plastics leach poisonous chemicals into the ground as they deteriorate, threatening global fresh water supplies and aquifers. Disposal of plastics by burning releases toxic byproducts of combustion into the air. And plastic items—especially bags—threaten marine life as they are often mistaken for squid and other prey.

Some countries have outlawed use of plastic bottles, bags and other items, recognizing their deleterious impact on the environment. Recycling programs aim to re-use plastic components, but as a species we are far from solving our dependence on plastic containers without continuing to harm the environment we live in.

It is believed that individual efforts are necessary to combat the global pollution problem that plastics present—in particular, utilizing containers for food and water that are easily capable of multiple use. However, washing alone introduces the problem of inadequate drying, which can lead to mold and bacteria formation. Molds can cause health issues if ingested or inhaled, and oftentimes it is difficult to completely dry many such items and storing them with moisture can lead to bacterial growth.

In addition, some of these containers, most notably narrow-mouth, reusable bottles and plastic bottles do not balance up-side down due to their thin neck and lightweight body; similarly, bags, notably plastic bags tend to collapse. The thin neck of the bottles can impede air flow, thus reducing the ability to quickly dry them before mold or other species take hold. Other popular bottles tend to have a top ridge where water tends to accumulate when merely tipped upside down to dry, thus requiring multiple revolutions and hours if not days of air drying. Quick drying also allows for decluttering of kitchen space where food preparation is needed.

Personal water bottles and thermoses have become increasingly popular accessories for their convenience, environmental benefits, and practicality. These containers offer an eco-friendly and efficient way to carry and consume beverages such as coffee, tea, water, etc. Many people re-use personal water bottles and thermoses, sometimes only rinsing the container between uses. In such cases, it becomes increasingly important to remove the contributors to mold and bacteria growth—in particular, moisture.

A drying rack system capable of quickly drying items, in particular bottles and the like, and optionally sterilizing the interior portions thereof is a long-felt need in the art of recycling and re-using household products.

### SUMMARY

In general, portable household drying rack system is disclosed. In one exemplary embodiment, a drying rack system includes one or more vertically standing arms. Each arm is capable of carrying an air current, supplied by a fan, that escapes at the end of the fan, along the arm of the fan, or both. Each arm is narrow enough to allow bottles, such as water bottles, bags, glassware and other items to be placed thereon, such that the arm is inserted into the item. A stream of air is forced into the bottle to achieve rapid drying. The air can be heated to facilitate even faster drying, and each arm can also include one or more sterilization features. One such sterilization feature is a source of ultraviolet light configured to emit an effective dose of electromagnetic radiation sufficient to cause cell death or disruption. In this way, bottles, bags, cups, glassware and any other reusable item capable of being placed on the arm can be dried and sterilized quickly and easily.

In one general aspect, a drying system includes a base, a fan assembly within the base capable of creating an air flow, and at least one elongate, hollow drying arm configured to receive an article to be dried, in communication with the air flow. The at least one drying arm includes at least one aperture allowing the air flow to escape therefrom. The article to be dried can be, for example, and without limitation, a bottle, bag, vase, glass or cup.

In one embodiment, the drying system further includes a heating element in communication with the air flow that provides a source of heated air to the article. The heating element can be housed within the base, for example.

In one embodiment, the drying system further includes an air filter in communication with the air flow that filters the air flow before reaching the at least one drying arm. The filter can be disposed on an exterior portion of the base.

In one embodiment, a top portion of each of the at least one drying arm is capped, and the at least one aperture is disposed proximal to the capped top portion.

In one embodiment, the airflow rate is adjustable. In an example, the system can further include an adjustable valve disposed between the fan and the at least one aperture of the at least one drying arm that modifies the airflow rate.

In one embodiment, the fan assembly includes a lower housing, the lower housing including an aperture for the airflow to flow therethrough, a powered fan, an upper housing including a fan aperture for each of the at least one drying arms, and a tube extending from the fan aperture configured to receive one of the at least one drying arms. The tube extending from the fan aperture can include a tube plate extending through a cross-section of the tube, the tube plate including at least one flow-reducing aperture. The drying arm can include a drying arm plate extending through a cross-section of the drying arm, the drying arm plate itself including an aperture. The drying arm can be rotatable relative to the tube, allowing the aperture to be aligned with one of the at least one flow-reducing aperture for controlling the amount of airflow passing through the drying arm.

In one embodiment, the drying system further includes at least one light source disposed on the drying arm, the light source being capable and configured to administer an effective dose of radiation to cause cell death or cell damage to mold, bacteria or viruses on the article.

In one embodiment, the drying system, further includes an accessory drying support that is configured to receive the airflow exiting the at least one drying arm. The drying support can include, for example, an attachment section configured to attach to the drying arm and receive the airflow and an extension configured to receive the airflow.

The extension can be, for example, U-shaped or branch into a plurality of fingers, each finger including a finger aperture allowing the airflow to escape therefrom.

In one embodiment, the drying system further includes an electronic logic control board, the logic control board being configured to receive at least one control input from a user, and control operation of the fan according to the at least one control input. The control input can be, for example, an adjustable timer for controlling a length of time that the fan operates before the logic control board deactivates the fan.

In a second general aspect, a drying assembly for drying articles includes a fan assembly capable of creating an airflow, a housing supporting the fan assembly, at least one hollow drying arm in communication with the airflow, the at least one hollow arm being configured to receive and support an article to be dried, the drying arm including an aperture allowing the airflow to escape therefrom, and a heating element in communication with, and for heating the airflow, wherein the airflow escaping the aperture is adjustable.

In one embodiment, the fan is a variable-speed fan capable of creating airflows of different rates.

In one embodiment, the drying assembly further includes a catch basin disposed between the fan assembly and the at least one drying arm for catching moisture drips from the article.

In a third general aspect, a drying assembly includes a housing, a variable-speed fan assembly disposed within the housing capable of, and configured to create an airflow from the surrounding environment, a plurality of hollow drying arms extending from the housing and configured to receive the airflow, wherein the plurality of hollow drying arms include a source of light capable of sterilizing of an inner portion of the article, a temperature-controllable heating element in-line with the airflow, and an electronic logic control board configured to control the operation of the heating element and the variable-speed fan.

Unless otherwise defined, all technical and scientific terms used herein have the same meaning as commonly understood by one of ordinary skill in the art. Although methods and materials similar or equivalent to those described herein can be used in the practice or testing of any described embodiment, suitable methods and materials are described below. In addition, the materials, methods, and examples are illustrative only and not intended to be limiting. In case of conflict with terms used in the art, the present specification, including definitions, will control.

The foregoing summary is illustrative only and is not intended to be in any way limiting. In addition to the illustrative aspects, embodiments, and features described above, further aspects, embodiments, and features will become apparent by reference to the drawings and the following detailed description and claims.

### DESCRIPTION OF DRAWINGS

The present embodiments are illustrated by way of the figures of the accompanying drawings, which may not necessarily be to scale, in which like references indicate similar elements, and in which:

FIG. 1 is a drying rack system, according to one embodiment;

FIG. 2 illustrates an article being dried on the drying rack system of FIG. 1;

FIG. 3 is a top portion of a fan assembly according to one embodiment;

FIG. 4 is an exploded view of a base portion of a drying rack system, according to one embodiment;

FIG. 5 is a top plan view of a lower portion of the base of a drying rack system, according to one embodiment;

FIG. 6 illustrates a drying arm with sanitizing light sources, according to one embodiment;

FIG. 7 illustrates plates for use in controlling air flow of the drying rack system according to one embodiment; and

FIGS. 8A and 8B illustrate drying arms according to two alternative embodiments.

### DETAILED DESCRIPTION OF ILLUSTRATIVE EMBODIMENTS

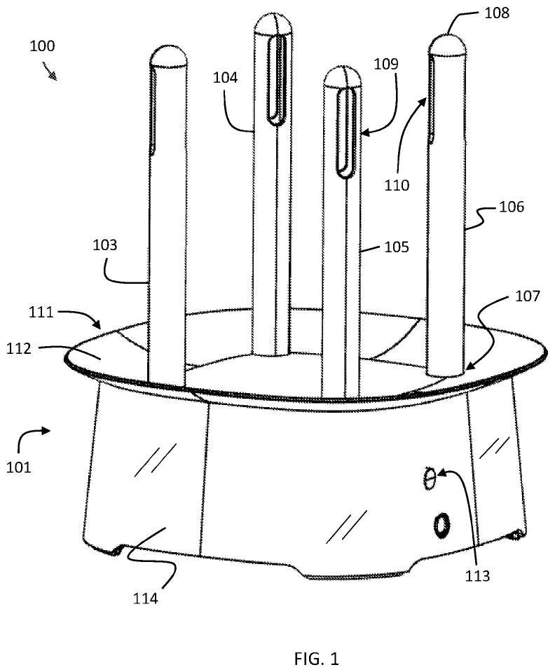

FIG. 1 is a drying rack system (hereinafter ‘system’) 100 according to one embodiment. In this embodiment, the system 100 includes a base 101 that houses at least one fan for delivering an airflow, an optional light source capable of producing a sterilizing light such as ultraviolet light, and control features for operating the system 100. Extending vertically from the base 101 in this embodiment are drying arms 103, 104, 105 and 106, each of which are configured to receive air flow from the at least one fan and light from the light source from the base 101. Each of the drying arms is configured to support various types of containers, for example, and without limitation, bottles, glasses, thermally insulted containers, bags, and others.

In this embodiment, each of the drying arms 103, 104, 105, 106 are tubular, having a first end portion (e.g., first end portion 107 of drying arm 106) that is open-ended to receive air flow from the at least one fan, and is configured to reversibly attach to the base 101. In this embodiment, each of the drying arms includes a second end portion (e.g., second end portion 108 of drying arm 106) opposite the first end portion. Each drying arm includes an air vent aperture (e.g., air vent apertures 109, 110 of drying arms 105, 106, respectively) near the second end portion of each drying arm as illustrated. The drying arms can be formed from plastic, metal or any other material that allows air flow therethrough as discussed in greater detail below.

In this embodiment, the base 101 includes a bowl portion 111 having an inclined side wall 112, for the purpose of catching and holding any drips from containers being dried on the drying arms. In this embodiment, each of the drying arms is secured to the base in a waterproof fashion so as to prevent any captured liquid from entering the interior of the base. For example, an O-ring (not shown in FIG. 1) can be positioned between the first end portion 107 of each drying arm and the portion of the base 101 where the drying arm is attached.

In this embodiment, the base 101 includes a timer controller 113 that is used to set a drying time; if the optional light source is utilized, the timer controller 113 can also control the amount of time that the light source is activated and deactivated. The timer controller 113 is in electronic communication with a control board (described below) and is configured to activate the one or more fans when the controller is turned to a selected timer value and deactivate the one or more fans (and, optionally the light source) when the timer counts down to zero. A protective exterior housing 114 protects the internal components of the base 101.

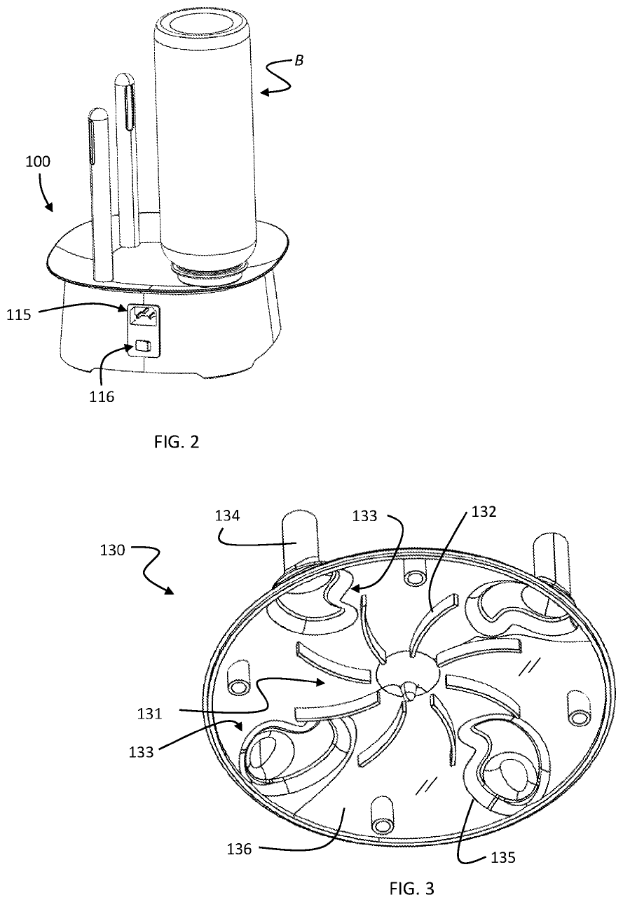

Referring now to FIG. 2, in this embodiment, the base 101 includes a power inlet 115 configured to receive an external electrical power source such as household alternating current (‘A/C’). A corded plug (not shown in FIG. 2) can connect the system 100 to external power via the power inlet 115. A switch 116 allows a user to toggle external power on or off from the power inlet 115 to the control board and other electronic components of the system while leaving the system 100 plugged in.

FIG. 2 illustrates an inverted bottle B being supported by one of the drying arms. In this example, the interior of bottle B receives an airflow from the one or more fans that is directed through the drying arm. The airflow causes the interior of the bottle B to dry faster than would otherwise occur if the bottle B were simply inverted and left to dry in an ambient environment.

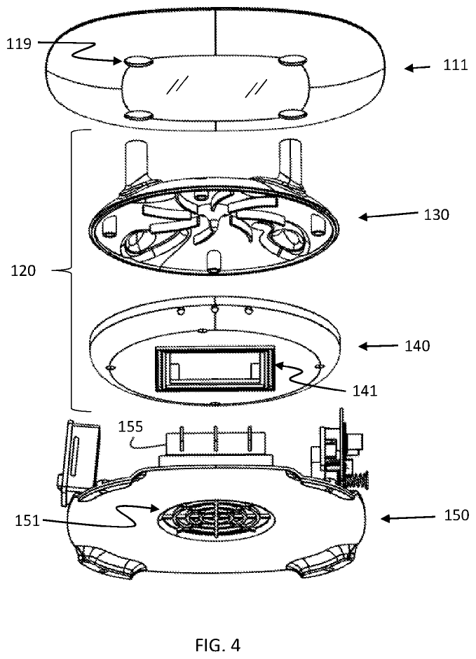

Referring now to FIGS. 3 and 4, select components of the system 100 are illustrated. FIG. 4 is an exploded view of select components of the base 101 with the protective exterior housing 114 removed for figure clarity. In this embodiment, the base 101 includes, inter alia, the bowl portion 111, a fan assembly 120, the fan assembly including top (130) and bottom (140) portions, and a bottom portion 150.

Referring to FIG. 3 in particular, in this embodiment, the top portion 130 of the fan assembly 120 includes an impeller assembly 131 having multiple fan blades 132. As the impeller rotates, the fan blades draw air from the bottom vent 151 of the bottom portion 150 and expels the air out through a plurality of apertures 133 in the top portion 130. Each aperture 133 leads to a vertically-extending tube 134, and each of the tubes 134 is configured to receive one of the drying arms (e.g., drying arm 103, 104, 105 or 106). The inner circumference of each drying arm is equal to, or slightly greater than the outer circumference of each tube 134, so that the drying arm can be slid onto the tube and held thereon by frictional engagement.

In this embodiment, the shape of each aperture 133 at its base 135, e.g., on the bottom surface 136 of the top portion 130, is “comma” shaped to maximize the amount of input air flow from the fan blades and to direct the forced air upward into the tube 134 with maximum efficiency. However, the apertures 133 may be any other shape, e.g., circular, oval-shaped, etc.

Referring back to FIG. 4, in this embodiment, the bowl portion 111 includes a plurality of apertures 119 equal to the number of drying arms of the system 100. Each aperture 119 is sized to accommodate fitting of each tube therethrough when the base is in an assembled configuration, e.g., as in FIG. 1. The bottom portion 140 of the fan assembly 120 includes a central aperture 141, allowing air to be drawn therethrough when the impeller 131 is activated.

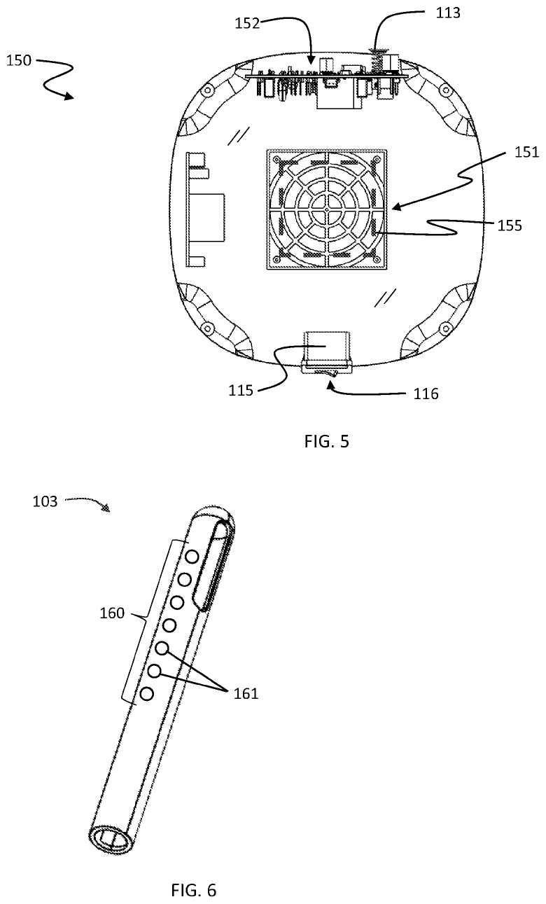

Referring now to FIG. 5, a top-view of the bottom portion 150 of the base 101 is shown in detail. In this embodiment, the bottom portion 150 includes a central aperture 151 through which air is drawn by the fan assembly 120 as previously described. A control board 152 is configured to receive power from the power inlet 115 when switched on by switch 116 and controls the operation of various components of the system. For example, the control board is configured to activate the fan assembly 120 either in an “on/off” mode, or by way of the timer controller 113 wherein a user can select a predetermined amount of drying time.

In this embodiment, a heating element 155 is positioned over the central aperture 151 (heating element 155 is illustrated as a dashed line in FIG. 5 for clarity of the drawing). In one approach, the heating element 155 is controlled by the control board and is activated when the fan is activated, heating the air as it is drawn through the central aperture 151 before it is sent into the drying arms. In a different approach, the heating element can have a separate exterior control, allowing a user to apply heat only when desired. The operating temperature of the heating element can be controlled by the user, e.g., from a low to high setting, providing variability in drying time. The heating element can be one that provides sufficient heat to provide a level of sterilization to the objects being dried.

In this embodiment, the bottom portion 150 of the base 101 can include a filter holder (not shown in the figures) for placing an air filter in-line with the central aperture 151. Accordingly, the air used to dry the objects can be filtered both to prevent dust, mold, and other materials from being blown into the objects and also to protect the fan assembly 120 from the same.

Referring now to FIG. 6, in this and other embodiments, each drying arm, e.g., drying arm 103 shown, can include one or arrays 160 of sterilizing light sources 161. The arrays 160 can be activated by the control board 152 from user control input or automatically when the fan is activated. The light sources 161 are capable of emitting a dose of radiation sufficient to cause cell death and/or damage to bacteria, viruses, molds, spores, and other organisms. One non-limiting example of this type of radiation is that in the ultraviolet portion of the spectrum, e.g., UV-C. In this and other embodiments, a removable safety cover can be fitted over each array of light sources when they are not utilized.

Still referring to FIG. 6, in this embodiment, the apertures (e.g., aperture 109, 110 of drying arms 105, 106, respectively) are positioned beneath the second end portion 108, e.g., the “top” of the drying arms. The second end portions 108 of the drying arms are dome-shaped to minimize the contact area of the drying arm with the object being dried. The displacement of the vent apertures from the top of the drying arms prevents lighter objects, such as plastic bags or plastic water bottles from being blown off the drying arm during drying operations.

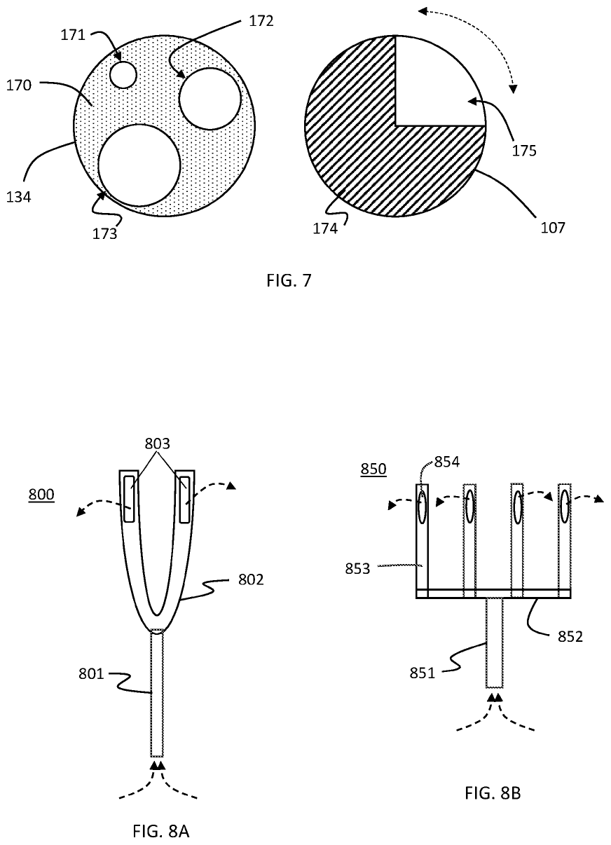

Referring now to FIG. 7, in this and embodiments, the flow rate of air exiting the drying arms can be adjustable. As one example, the left portion of FIG. 7 depicts a cross-sectional view of tube 134 according to one alternative embodiment. As previously described, tube 134 extends from a top portion of the fan assembly 120. The right portion of FIG. 7 depicts a cross-sectional view of a drying arm, e.g., drying arm 107 according to one alternative embodiment. In these embodiments, the tube 134 includes a solid plate 170 spanning the cross-sectional diameter of the tube 134. The shelf includes three apertures, 171, 172 and 173, each increasing in aperture size, respectively. It should be understood that more or fewer apertures can be utilized in the present embodiments. Drying arm 107 also includes a solid cross-sectional plate 174, the plate having a wedge-shaped aperture 175 as illustrated.

In use, the drying arm 107 fits over the tube 134 as previously described; in this embodiment, when the drying arm 107 is placed over the tube 134, the plates 170, 174 are in confrontation with one another and coaxially aligned. To change the flow rate of air, the drying arm 107 can be rotated (as illustrated by the dashed double-headed arrow) so that the aperture 175 aligns with the small (171), medium (172) or large (173) aperture, or a combination thereof.

It should be understood that other approaches can be used for controlling the flow rate of air, such as employing a slidable plate in the drying arms that can be shifted from a fully-open configuration to a fully-blocked configuration.

Referring to FIGS. 8A and 8B, alternative drying arms are illustrated according to several embodiments. For example, in the embodiment of FIG. 8A, the drying arm 800 includes a main riser tube 801 connected to a U-shaped tube 802. At the top of the “U”, two apertures 803 are disposed. In this embodiment, air is drawn up through the main riser tube 801 as depicted by the upward-pointing dashed arrows and expelled out of the two apertures 803 as illustrated. The U-shaped drying arm 800 of FIG. 8A can be used, for example, for drying plastic bags and the like for re-use. In an alternative embodiment, a U-shaped tube adaptor, similar to that shown in FIG. 8A can be configured to fit over the top of any of the drying arms, e.g., drying arms 103-106 so that a U-shaped drying arm can be used without requiring the drying arms to be removed.

In another example, FIG. 8B illustrates a branched drying arm 850. In this example, the drying arm 850 includes a main riser tube 851, which leads to a distribution tube 852. Four branch arms 853, each having an aperture 854 located near the top of each arm are connected to the distribution tube 852. In use, air is drawn up the main riser tube 851 and is distributed to each branch arm 853 by way of the distribution tube 852. The branched trying arm 850 can also be used for drying plastic bags or any other item which would benefit from being held in an open configuration to maximize drying efficiency and reduce areas where moisture may otherwise accumulate. In an alternative embodiment, a branched drying arm adaptor, similar to that shown in FIG. 8B can be configured to fit over the top of any of the drying arms, e.g., drying arms 103-106 so that a branched drying arm can be utilized without requiring the drying arms to be removed.

In both embodiments shown in FIGS. 8A and 8B, the main riser tube 801, 851 can be connected to tube 134 of top portion 130 of the fan assembly 120 as described previously with respect to the drying arms, e.g., drying arm 103.

In general, a method of drying and sterilizing an article includes first washing the article, e.g., by hand. Next, the fan assembly 120 can be activated by activating the power supply of the control board and either manually turning the fan “on” or setting an appropriate timer by way of the external user controls in signal connection with the control board. Next, the article can be placed upon one or more of the drying arms 103-106. The air that blows through the vertical arms is directed into the interior of the article, accelerating the drying process. At the same time, the array of light sources 160 can be optionally activated, if so equipped, to ensure that any microorganisms present inside the article are destroyed or are made incapable of proliferation.

A number of illustrative embodiments have been described. Nevertheless, it will be understood that various modifications may be made without departing from the spirit and scope of the various embodiments presented herein. For example, the embodiment shown in FIGS. 1 and 2 include four (4) vertical arms; however, the concepts described herein can be expanded to include a system having six, eight, ten or any number of vertical arms. The arms can be configured to accept any size bottle, can, bag, glass, vase or other containers and storage vessels. Accordingly, other embodiments are within the scope of the following claims.

1. A drying system, comprising:
a base;
a fan assembly within said base capable of creating an air flow; and
at least one elongate, hollow drying arm configured to receive an article to be dried, in communication with said air flow;
wherein said air flow is adjustable by way of an adjustable valve disposed between said fan and said at least one aperture of said at least one drying arm that modifies said air flow rate; and
wherein said at least one drying arm comprises at least one aperture allowing said air flow to escape therefrom.

2. The drying system of claim 1, further comprising a heating element in communication with said air flow that provides a source of heated air to said article.

3. The drying system of claim 2, wherein said heating element is housed within said base.

4. The drying system of claim 1, further comprising an air filter in communication with said air flow that filters said air flow before reaching said at least one drying arm.

5. The drying system of claim 4, wherein said filter is disposed on an exterior portion of said base.

6. The drying system of claim 1, wherein said article to be dried is glassware, a bottle, bag, vase, glass or cup.

7. The drying system of claim 1, wherein a top portion of each of said at least one drying arms is capped, and wherein said at least one aperture is disposed proximal to said capped top portion.

8. (canceled)

9. (canceled)

10. (canceled)

11. (canceled)

12. The drying system of claim 1, further comprising at least one light source disposed on said drying arm, said light source being capable and configured to administer an effective dose of radiation to cause cell death or cell damage to mold, bacteria or viruses on said article.

13. The drying system of claim 1, further comprising an accessory drying support that is configured to receive said airflow exiting said at least one drying arm.

14. The drying system of claim 13, wherein said drying support comprises:
an attachment section configured to attach to said drying arm and receive said airflow; and
an extension configured to receive said airflow;
wherein said extension is U-shaped or branches into a plurality of fingers, each finger comprising a finger aperture allowing said airflow to escape therefrom.

15. The drying system of claim 1, further comprising an electronic logic control board, said logic control board being configured to receive at least one control input from a user, and control operation of said fan according to said at least one control input.

16. The drying system of claim 15, wherein said control input is an adjustable timer for controlling a length of time that the fan operates before said logic control board deactivates said fan.

17. (canceled)

18. (canceled)

19. (canceled)

20. (canceled)

21. A drying system, comprising:
a base
a fan assembly within said base capable of creating an air flow; and
at least one elongate, hollow drying arm configured to receive an article to be dried, in communication with said air flow;
wherein said at least one drying arm comprises at least one aperture allowing said air flow to escape therefrom;
wherein said fan assembly comprises:
a lower housing comprising an aperture for said airflow to flow therethrough;
a powered fan;
an upper housing comprising a fan aperture for each of said at least one drying arms; and
a tube extending from said fan aperture configured to receive one of said at least one drying arms;
wherein said tube extending from said fan aperture comprises a tube plate extending through a cross-section of said tube, said tube plate comprising at least one flow-reducing aperture;
said drying arm comprises a drying arm plate extending through a cross-section of said drying arm, said drying arm plate comprising an aperture; and
wherein said drying arm is rotatable relative to said tube, allowing said aperture to be aligned with one of said at least one flow-reducing aperture for controlling the amount of airflow passing through said drying arm.

22. A drying and sterilizing apparatus comprising:
a base defining an internal air chamber and including a fan configured to generate airflow into the air chamber;
a plurality of drying arms, each drying arm being removably attachable to a corresponding outlet port in the base, each drying arm defining an internal conduit in fluid communication with the air chamber when attached;
wherein at least two of the drying arms differ in shape or configuration to accommodate different types of articles to be dried; and
a sterilization element associated with at least one of the drying arms or outlet ports, the sterilization element configured to sterilize an article placed over the drying arm.

23. The apparatus of claim 22, wherein at least one of the drying arms includes an internal passage that bifurcates to direct airflow through two separate outlet paths configured to simultaneously dry two regions of an article.

24. The apparatus of claim 22, wherein at least one of the drying arms includes a coupling mechanism for receiving an interchangeable nozzle or extension configured for insertion into an interior of the article to be dried.

25. The apparatus of claim 22, wherein the sterilization element comprises a light source configured to emit ultraviolet (UV) light toward an exterior surface of the article during drying.

26. The apparatus of claim 22, wherein the drying arms are configured for friction-fit insertion into the outlet ports of the base and are removable without the use of tool.

27. The apparatus of claim 22, wherein the drying arms comprise a curved or U-shaped conduit portion configured to conform to a selected shape of article.

28. The apparatus of claim 22, wherein the base includes a heating element configured to elevate the temperature of the airflow delivered to the drying arms.
